(19) United States
(12) Patent Application Publication
(10) Pub. No.: US 2022/0340887 A1
(43) Pub. Date: Oct. 27, 2022

(54) Polymerases

(71) Applicant: Sequlite Genomics US, Inc., Dublin, CA (US)

(72) Inventors: MARIAN PERIS, Redwood City, CA (US); CHRIS BENOIT, Jupiter, FL (US); MONITA SIENG, San Jose, CA (US)

(21) Appl. No.: 17/760,920

(22) PCT Filed: Sep. 16, 2020
(86) PCT No.: PCT/US20/51125

§ 371 Date: Mar. 16, 2022

Related U.S. Application Data
(60) Provisional application No. 62/902,351, filed on Sep. 18, 2019.

(51) Int. Cl. C12N 9/12; C07K 16/00

(52) U.S. Cl. CPC C12N 9/1241; C07K 16/00

(57) ABSTRACT

Modified polypeptides having polymerase activity.

### CROSS-REFERENCE TO RELATED APPLICATIONS

This application is a U.S. national stage application of PCT/US2020/51125, filed on Sep. 16, 2020, and published as WO 2021/055514 on Mar. 25, 2021, which is a continuation-in-part of and claims the benefit of priority of U.S. provisional application Ser. 62/902,351, filed Sep. 18, 2019, the contents of both of which are incorporated herein in its entirety.

### TECHNICAL FIELD

Modified viral enzymes.

### SUMMARY OF THE INVENTION

The present invention provides modified polypeptides having polymerase activity.

### BRIEF DESCRIPTION OF THE DRAWINGS

FIG. 1 shows a stained SDS-PAGE gel showing molecular weight markers (L), partially purified sample loaded (LD) onto a heparin column, with eluted fractions 8 through 16.

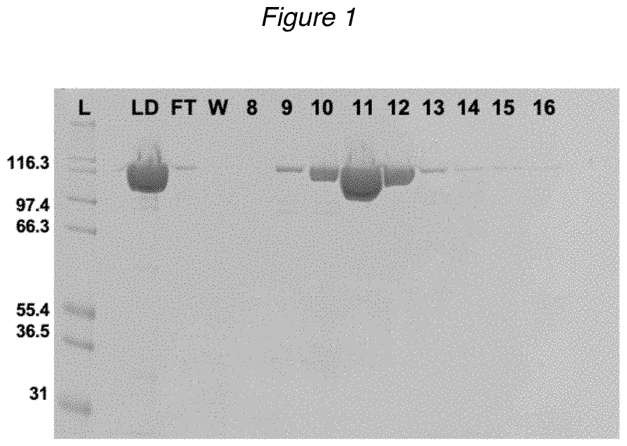

### DETAILED DESCRIPTION OF THE INVENTION

The present invention provides polypeptides that have one or more of the following modifications to SEQ ID NO:1, which has 903 designated amino acid positions.

At position 114, native Asp can be modified to Ala (D114A) or Val (D114V) to reduce activity in the exonuclease domain. It can also be modified to other aliphatic amino acids (such as G, L, I), or small amino acids (such as S or C), or N or T.

At position 115, native Ile (I115) can be modified to other aliphatic amino acids such as Leu (I115L) or Val (I115V), or to other aliphatic or hydrophobic residues such as F, M, T, W, or Y. Position 115 can also be modified to G or A.

At position 116, native Glu can be modified to Ala (E116A) or Val (E116V), also to reduce exonuclease activity. It can also be modified to other aliphatic amino acids (such as G, L, I), or small amino acids (such as G, S, C), or N or T.

At position 230, or 231 to 235, one of the amino acids can be modified to Ser or Cys.

At position 327, native Asp (D327) can be modified to Ala (D327A), Val (D327V), or to E, N, Q, or to other polar amino acids.

At position 415, native Leu can be modified to Tyr (L415Y), Ser (L415S) or Ala (L415A) to open up the nucleotide pocket area. It can be modified to S or aromatic amino acids like F or W. It can also be modified to H, T, M, or aliphatic amino acids.

At position 416, native Tyr can be modified to Ala (Y416A), Leu (Y416L) or Val (Y416V), which can increase the space for nucleotide substrates with bulky 3′ groups. It can also be modified to other aliphatic amino acids (such as G, V, I), or small amino acids (such as G, S, C), or N or T.

At position 417, native Pro can be modified to Val (P417V), Ile (P417I) or Ser (P417S) to accommodate 3′ groups. It can also be modified to aliphatic amino acids like G, A, L, I, or to M, F, C, R, D, or T.

At positions 557, the amino acids Ile may be modified to Gly (I557G), Lys (I557K), Ser (I557S), or Thr (I557T).

At position 558, native Asn can be modified to Ala (N558A). It can also be modified to Leu (N558L) or Val (N558V) or other aliphatic amino acids (such as G or I), or small amino acids (such as G, S, C), or N or T.

At position 559, the amino acid Arg may be modified to Asp (R559D), His (R559H), or Asn (R559N).

At position 587, native Thr can be modified to Ser (T587S) or Ala (T587A) It can also be modified to G, C, A or other small amino acids such as P, N, D, or V.

At position 594, native Leu can be modified to Ile (L594I). It can also be modified to V, M, C, A, or G.

At position 703, native Thr can be modified to Ile (T703I). It can also be modified to V, M, C, A, or G.

The amino acids at the modified positions can be chemically modified with phosphorylation, methylation, acetylation, amidation, formation of pyrrolidone carboxylic acid, isomerization, hydroxylation, sulfation, flavin-binding, cysteine oxidation, or nitrosylation.

The polypeptide of the invention can join a nucleotide (such as a naturally occurring or modified nucleotide) to a nucleic acid strand under suitable reaction conditions. The nucleotide can be modified at the 3′-OH position, such as with an allyl or azido group. The polypeptide can be full length, more or less than 903 amino acids, or contain an enzymatically active portion thereof, while retaining the position designations used for SEQ ID NO:1.

The polypeptide can contain additional sequences or sequence modifications to facilitate cloning, expression, or attachment for purification. For example, the amino-terminal amino acids can be modified to or augmented with Met-Ile-Leu, Asp-Thr-Asp, or a combination thereof. The Ile in the Met-Ile-Leu can be further modified to Ala-Val.

The invention encompasses polypeptides that have the modifications above, plus another 1, 2, 3, 4, 5, 6, 7, 8, 9, 10, 11, 12, 13, 14, 15, 16, 17, 18, 19, 20 different amino acids, or differ by 2, 2.5, 3, 3.5, 4, 4.5, 5, 5.5, 6, 6.5, 7, 7.5, 8, 8.5, 9, 9.5, 10, 11, 12, 13, 14, 15, 16, 17, 18, 19, or 20 percent.

The invention also provides nucleic acids that encode the polypeptides (or enzymatically active portions thereof) of the invention, as well as vectors having such nucleic acids or their complements. The codons of the nucleic acid sequences can be modified to optimize expression with the species of the desired system. The invention also provides polypeptides obtained by expressing such nucleic acids, and kits thereof.

Further provided are antibodies against the polypeptides, particularly detectably labeled antibodies. The invention includes methods for selectively detecting the presence of the polypeptide and methods for purifying polypeptides with antibodies conjugated to solid phase substrates.

### EXAMPLES

### Example 1: Modified Polypeptide

Polypeptides are prepared having SEQ ID NO:1 except modified at positions 114, 116, 415, 416, 417, and 558. The polypeptides are expressed from nucleic acid sequences that were prepared to encode the modified polypeptides.

### Example 2: Enzyme Purification and Activity

An enzyme was expressed having SEQ ID NO:1 modified with D114A, E116A, L415Y, Y416A, P417V, N558A, with a His-tag to facilitate purification. The enzyme was captured with a nickel-chelating column and loaded onto a Q Sepharose anion-exchange column. The eluted fractions were further purified using a heparin column. Fractions 10 to 12 show the purified 104 kDa enzyme with >99% purity.

The enzyme demonstrated dNTP incorporation at 30° C. to 39° C., with peak activity at 37° C. The enzyme also demonstrated incorporation of certain modified nucleotides.

The headings provided above are intended only to facilitate navigation within the document and should not be used to characterize the meaning of one portion of text compared to another. Skilled artisans will appreciate that additional embodiments are within the scope of the invention. The invention is defined only by the following claims; limitations from the specification or its examples should not be imported into the claims.

We claim:

1. A polypeptide having SEQ ID NO:1 and a modification of at least one of positions 114, 116, 415, 416, 417, or 558, and able to incorporate a nucleotide into a nucleic acid chain.

2. The polypeptide of claim 1, wherein the nucleotide is modified, optionally at the 3′-OH position.

3. The polypeptide of claim 1, having a mutation at 114, optionally 114A or 114V.

4. The polypeptide of claim 1, having a mutation at 116, optionally 116V or 116A.

5. The polypeptide of claim 1, having a mutation at 415, optionally 415A, 415S, or 415Y.

6. The polypeptide of claim 1, having a mutation at 416, optionally 416A, 416L, or 416V.

7. The polypeptide of claim 1, having a mutation at 417, optionally 417V, 417I, or 417S.

8. The polypeptide of claim 1, having a mutation at 558, optionally 558A, 558L, or 558V.

9. The polypeptide of claim 1, having modifications of at least four of positions 114, 116, 415, 416, 417, and 558.

10. The polypeptide of claim 1, further comprising a modification at position 115, 230, 231, 327, 587, 594, or 703.

11. The polypeptide of claim 1, further comprising at least one modification at 557, 558, or 559.

12. A polynucleotide encoding the polypeptide of claim 1.

13. An antibody that specifically binds to the polypeptide of claim 1.


SEQUENCE LISTING (1 sequence)

SEQ ID NO: 1 - protein, 903 aa, Unknown
  Description of Unknown Escherichia virus RB69
MKEFYLTVEQIGDSIFERYIDSNGRERTREVEYKPSLFAHCPESQATKYFDIYGKPCTRKLFANMRDASQWIKRMEDIGLEALGMDDFKLAYLSDTYNYEIKYDHTKIRVANFDIEVTSPDGFPEPSQAKHPIDAITHYDSIDDRFYVFDLLNSPYGNVEEWSIEIAAKLQEQGGDEVPSEIIDKIIYMPFDNEKELLMEYLNFWQQKTPVILTGWNVESFDIPYVYNRIKNIFGESTAKRLSPHRKTRVKVIENMYGSREIITLFGISVLDYIDLYKKFSFTNQPSYSLDYISEFELNVGKLKYDGPISKLRESNHQRYISYNIIDVYRVLQIDAKRQFINLSLDMGYYAKIQIQSVFSPIKTWDAIIFNSLKEQNKVIPQGRSHPVQPYPGAFVKEPIPNRYKYVMSFDLTSLYPSIIRQVNISPETIAGTFKVAPLHDYINAVAERPSDVYSCSPNGMMYYKDRDGVVPTEITKVFNQRKEHKGYMLAAQRNGEIIKEALHNPNLSVDEPLDVDYRFDFSDEIKEKIKKLSAKSLNEMLFRAQRTEVAGMTAQINRKLLINSLYGALGNVWFRYYDLRNATAITTFGQMALQWIERKVNEYLNEVCGTEGEAFVLYGDTDSIYVSADKIIDKVGESKFRDTNHWVDFLDKFARERMEPAIDRGFREMCEYMNNKQHLMFMDREAIAGPPLGSKGIGGFWTGKKRYALNVWDMEGTRYAEPKLKIMGLETQKSSTPKAVQKALKECIRRMLQEGEESLQEYFKEFEKEFRQLNYISIASVSSANNIAKYDVGGFPGPKCPFHIRGILTYNRAIKGNIDAPQVVEGEKVYVLPLREGNPFGDKCIAWPSGTEITDLIKDDVLHWMDYTVLLEKTFIKPLEGFTSAAKLDYEKKASLFDMFDF